(19) United States
(12) Patent Application Publication
(10) Pub. No.: US 2024/0257901 A1
(43) Pub. Date: Aug. 1, 2024

(54) INFORMATION PROCESSING DEVICE, INFORMATION PROCESSING METHOD, AND NON- TRANSITORY COMPUTER-READABLE STORAGE MEDIUM STORED WITH INFORMATION PROCESSING PROGRAM

(71) Applicant: TOYOTA JIDOSHA KABUSHIKI KAISHA, Toyota-shi (JP)

(72) Inventors: Masao YANO, Sunto-gun (JP); Hanae SHIRATORI, Susono-shi (JP); Yasuhiro TOYAMA, Kariya-shi (JP)

(73) Assignee: TOYOTA JIDOSHA KABUSHIKI KAISHA, Toyota-shi (JP)

(21) Appl. No.: 18/403,907

(22) Filed: Jan. 4, 2024

(30) Foreign Application Priority Data
Jan. 30, 2023 (JP) 2023-012269

(51) Int. Cl. G16B 15/00; G16B 40/20; G16B 45/00

(52) U.S. Cl. CPC G16B 15/00; G16B 40/20; G16B 45/00

(57) ABSTRACT

An information processing device, including: a memory, and a processor coupled to the memory, wherein the processor is configured to: generate an atom-coordinate image expressing atomic coordinates in a molecule; perform a Fourier transformation on the atom-coordinate image to produce power spectrum data; perform principal component analysis on the power spectrum data so as to derive, from the power spectrum data, principal component vectors expressing basis vectors of the power spectrum data and principal component scores expressing contained quantities of the principal component vectors; derive index values expressing degrees of correlation between the principal component scores and a performance of the molecule; identify any principal component vectors that correlate with the molecule performance based on the index values; and output principal component power spectrum data that is power spectrum data corresponding to the principal component vectors.

### CROSS-REFERENCE TO RELATED APPLICATION

This application is based on and claims priority under 35 USC 119 from Japanese Patent Application No. 2023-012269 filed on Jan. 30, 2023, the disclosure of which is incorporated by reference herein.

### BACKGROUND

### Technical Field

The present disclosure relates to an information processing device, an information processing method, and a non-transitory computer-readable storage medium stored with an information processing program.

### Related Art

For example, a method for searching for novel materials is described in Japanese Patent Application Laid-Open (JP-A) No. 2017-091526. This method includes a stage of performing training on a material model that has been modeled based on a known material, a stage of inputting a target physical property and deciding at least one candidate material in results of training, and a stage of deciding a novel material from out of the at least one candidate materials.

In the technology described in JP-A No. 2017-091526, training is performed by machine learning of a relationships between structure information and the physical property information of known materials, and deciding at least one candidate material by inputting the target physical property into the learned model obtained thereby.

However, when searching for molecules to configure materials in the above technology, known technology of extended connectivity circular fingerprints (ECFP), RDKit, or the like is employed to investigate relationships between feature values and performance of molecules, and molecules with a possibility to satisfy a performance condition are compared against a database. This means that it is not possible to search for molecules other than those already stored in the database.

### SUMMARY

The present disclosure provides an information processing device, an information processing method, and an information processing program that are capable of raising the possibility of discovering an unknown molecule that satisfy a performance condition compared to cases in which referencing is performed against a database.

An information processing device of a first aspect of the present disclosure includes a generation section that generates an atom-coordinate image expressing atomic coordinates in a molecule, a spectrum production section that performs a Fourier transformation on the atom-coordinate image to produce power spectrum data, a principal component derivation section that performs principal component analysis on the power spectrum data so as to derive from the power spectrum data principal component vectors expressing basis vectors of the power spectrum data and principal component scores expressing contained quantities of the principal component vectors, an index value derivation section that derives index values expressing degrees of correlation between the principal component scores and a performance of the molecule, an identification section that identifies any principal component vectors that correlate with the molecule performance based on the index values, and an output section that outputs principal component power spectrum data that is power spectrum data corresponding to the identified principal component vectors.

The first aspect of the present disclosure is able to raise a possibility of discovering an unknown molecule that satisfies a performance condition compared to cases in which referencing is performed against a database.

An information processing device of a second aspect of the present disclosure is the information processing device of the first aspect of the present disclosure, wherein the index value derivation section derives the index values using a learned model that has undergone machine learning in advance so as to output the molecule performance in response to being input with the principal component scores. A configuration may be adopted in which plural sets of training data are prepared in which principal component scores have been associated with molecule performance, a learned model is generated based on the plural sets of training data, and the learned model is utilized to index values. In such cases a known machine learning model may be employed as the learned model. The learned model may, for example, be generated by training the machine learning model using a deep learning algorithm.

The second aspect of the present disclosure is able to derive index values with good accuracy by using the learned model.

An information processing device of a third aspect of the present disclosure is the information processing device of the first aspect or the second aspect of the present disclosure, wherein the generation section generates the atom-coordinate image in two-dimensions or in three-dimensions from a molecule data file stored with information representing a structure of the molecule.

The third aspect of the present disclosure is able to impose position conditions on atoms in two-dimensions or three-dimensions.

The information processing device of the fourth aspect of the present disclosure is the information processing device of any one of the first aspect to the third aspect, wherein the identification section identifies plural of principal component vectors, and the information processing device further includes a map generation section that generates two-dimensional map data in which the principal component scores corresponding to each of the plural principal component vectors are projected as plural plot points onto two-dimensions.

The fourth aspect of the present disclosure enables correspondence relationships between principal components having a high correlation to the molecule performance to be expressed as the two-dimensional map.

An information processing device of a fifth aspect of the present disclosure is the information processing device of the fourth aspect of the present disclosure, wherein the output section displays the two-dimensional map data together with the atom-coordinate image corresponding to the plot points of the two-dimensional map data at a display section.

The fifth aspect of the present disclosure enables a user to understand the atom-coordinate image corresponding to the plot points.

Furthermore, an information processing method of a sixth aspect of the present disclosure is performed by an information processing device generating an atom-coordinate image expressing atomic coordinates in a molecule, performing a Fourier transformation on the atom-coordinate image to produce power spectrum data, performing principal component analysis on the power spectrum data so as to derive from the power spectrum data principal component vectors expressing basis vectors of the power spectrum data and principal component scores expressing contained quantities of the principal component vectors, deriving index values expressing degrees of correlation between the principal component scores and a performance of the molecule, identifying any principal component vectors that correlate with the molecule performance based on the index values, and outputting principal component power spectrum data that is power spectrum data corresponding to the identified principal component vectors.

The sixth aspect of the present disclosure, similarly to the first aspect, is able to raise the possibility of discovering an unknown molecule that satisfies a performance condition compared to cases in which referencing is performed against a database.

Furthermore, an information processing program of a seventh aspect of the present disclosure causes processing to be executed by a computer. The processing includes generating an atom-coordinate image expressing atomic coordinates in a molecule, performing a Fourier transformation on the atom-coordinate image to produce power spectrum data, performing principal component analysis on the power spectrum data so as to derive from the power spectrum data principal component vectors expressing basis vectors of the power spectrum data and principal component scores expressing contained quantities of the principal component vectors, deriving index values expressing degrees of correlation between the principal component scores and a performance of the molecule, identifying any principal component vectors that correlate with the molecule performance based on the index values, and outputting principal component power spectrum data that is power spectrum data corresponding to the identified principal component vectors.

The seventh aspect of the present disclosure, similarly to the first aspect, is able to raise the possibility of discovering an unknown molecule that satisfies a performance condition compared to cases in which referencing is performed against a database.

As described above, the present disclosure exhibits the excellent advantageous effect of enabling the possibility of discovering an unknown molecule that satisfies a performance condition to be raised compared to cases in which referencing is performed against a database.

### BRIEF DESCRIPTION OF THE DRAWINGS

Exemplary embodiments of the present disclosure will be described in detail based on the following figures, wherein:

FIG. 1 is a block diagram illustrating an example of a configuration of an information processing system according to an exemplary embodiment;

FIG. 2 is a block diagram illustrating an example of a functional configuration of a server according to an exemplary embodiment;

FIG. 3A is a diagram illustrating an example of molecule data files according to an exemplary embodiment;

FIG. 3B is a diagram illustrating an example of atom-coordinate images according to an exemplary embodiment;

FIG. 4 is a diagram to accompany explanation of how a power spectrum is made of atom-coordinate images according to an exemplary embodiment:

FIG. 5 diagram to accompany explanation of featurization of waveform data according to an exemplary embodiment;

FIG. 6A is a graph illustrating an example of plot points plotted for true values and prediction values related to molecule performance as output from a learned model according to an exemplary embodiment;

FIG. 6B is a diagram illustrating an example of index values derived for each principal component vector using molecule performance obtained from a learned model;

FIG. 6C is a diagram illustrating an example of index values derived for each principal component vector using molecule performance obtained from a learned model;

FIG. 6D includes graphs illustrating examples of principal component power spectrum data corresponding to principal component vectors:

FIG. 7A is a graph illustrating an example of power spectrum data prior to changing principal component scores of principal components PC1 and PC3;

FIG. 7B is a graph illustrating an example of power spectrum data after changing principal component scores of principal components PC1 and PC3;

FIG. 8 is a diagram illustrating an example of a two-dimensional map data and atom-coordinate images according to an exemplary embodiment;

FIG. 9 is a diagram to accompany explanation of prediction using molfile structure information according to the present exemplary embodiment; and

FIG. 10 is a flowchart illustrating an example of a flow of processing by an information processing program according to an exemplary embodiment.

### DETAILED DESCRIPTION

Detailed description follows regarding an example of an exemplary embodiment to implement technology disclosed herein, with reference to the drawings. Note that the same reference numerals are allocated across all the drawings to configuration elements and processing with behavior, operation, and function performing the same role, and sometimes duplicate description thereof is omitted as appropriate. Each of the drawings is merely a schematic illustration to enable sufficient understanding of the technology disclosed herein. The technology disclosed herein is accordingly not limited to only the examples illustrated. Note that sometimes explanation is omitted in the present exemplary embodiment for configuration not directly related to the present disclosure and for known configuration.

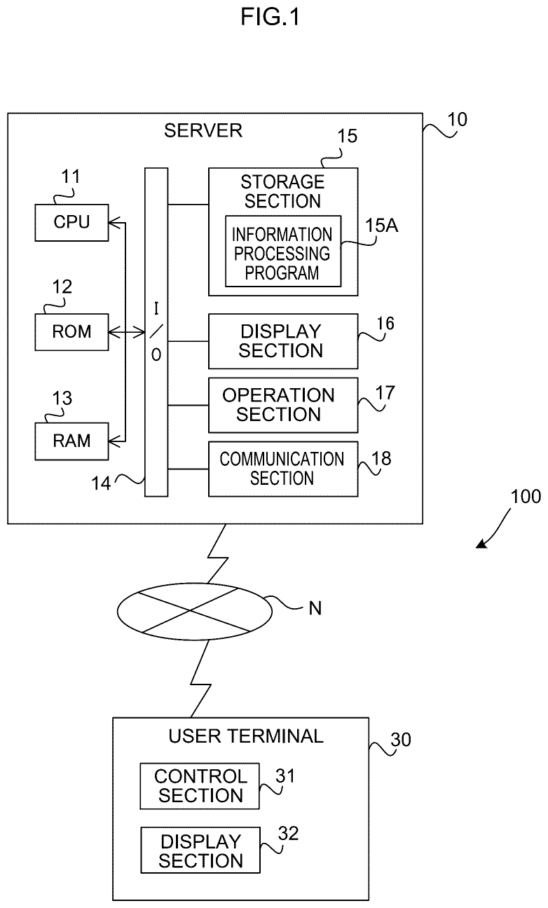

FIG. 1 is a block diagram illustrating an example of a configuration of an information processing system 100 according to the present exemplary embodiment.

As illustrated in FIG. 1, the information processing system 100 according to the present exemplary embodiment includes a server 10 and a user terminal 30. The server 10 is an example of an information processing device. The server 10 and the user terminal 30 are connected so as to be able to communication over a network N.

The server 10 includes a central processing unit (CPU) 11, read only memory (ROM) 12, random access memory (RAM) 13, an input/output interface (I/O) 14, a storage section 15, a display section 16, an operation section 17, and a communication section 18. The server 10 is, for example, configured as a general purpose computer device.

The CPU 11, the ROM 12, the RAM 13, and the I/O 14 are each connected together through a bus. Each functional section including the storage section 15, the display section 16, the operation section 17, and the communication section 18 is connected to the I/O 14. Each of these functional sections is able to communicate with the CPU 11 through the I/O 14.

A control section is configured by the CPU 11, the ROM 12, the RAM 13, and the I/O 14. The control section may be configured as a sub-control section to control operation of part of the server 10, and may be configured as a part of a main control section to control overall operation of the server 10. An integrated circuit such as a large scale integration (LSI) or the like or an IC chip set is, for example, employed for part of or all of each block of the control section. A separate individual circuit may be employed for each of the above blocks, and a circuit that integrates part or all thereof may be employed therefor. Each of the above blocks may be provided as a single body, and some of the blocks may be provided separately. Moreover, part of each of the blocks may be provided separately. The integration of the control section is not limited to LSI, and a dedicated circuit or a general purpose processor may be employed.

The storage section 15 employs, for example, a hard disk drive (HDD), an solid state drive (SSD), a flash memory or the like. An information processing program 15A according to the present exemplary embodiment is stored in the storage section 15. Note that the information processing program 15A may be stored on the ROM 12.

The information processing program 15A is, for example, a program pre-installed on the server 10. The information processing program 15A may be stored on anon-transitory storage medium, or may be implemented by being distributed over the network N and appropriately installed on the server 10. Note that conceivable examples of the non-transitory storage medium include a compact disk read only memory (CD-ROM), a magneto-optical disc, an HDD, a digital versatile disc read only memory (DVD-ROM), flash memory, a memory card, and the like.

The display section 16 employs, for example, a liquid crystal display (LCD), an organic electro luminescence (EL) display, or the like. The display section 16 may include an integrated touch panel. The operation section 17 is, for example, provided by a device such as a keyboard, mouse, or the like for use in operational input. The display section 16 and the operation section 17 receive various instructions from a user of the server 10. The display section 16 displays various information such as the result of processing executed according to instructions received from a user, and notifications and the like for processing.

The communication section 18 is, for example, connected to a network N such as the internet, a local area network (LAN), a Wide Area Network (WAN), or the like, and is able to communicate with the user terminal 30 over the network N.

The user terminal 30 is operated by a user. The user terminal 30 includes, from a functional perspective, a control section 31 and a display section 32, as illustrated in FIG. 1.

The control section 31 controls operation of the user terminal 30. The display section 32 displays various information according to control by the control section 31.

The server 10 of the information processing system 100 according to the present exemplary embodiment generates atom-coordinate images representing atomic coordinates in a molecule so as to derive later-described principal component vectors and principal component scores, performs a Fourier transformation on the atom-coordinate images so as to produce power spectrum data, and performs principal component analysis on the power spectrum data. The server 10 of the information processing system 100 employs a learned model to derive index values expressing a degree of correlation between the principal component scores and molecule performance, identifies any principal component vectors having a comparatively high correlation to the molecule performance based on the index values, and outputs principal component power spectrum data that is power spectrum data corresponding to the identified principal component vectors. Doing so enables an understanding of the shape (power spectrum) of the principal components, and enables clarification of requirements demanded in a molecule structure. Namely, a possibility can be raised of discovering an unknown molecule (unknown structure) that satisfies a performance condition by imposing atom position conditions using an atom-coordinate image instead of by comparison against a database.

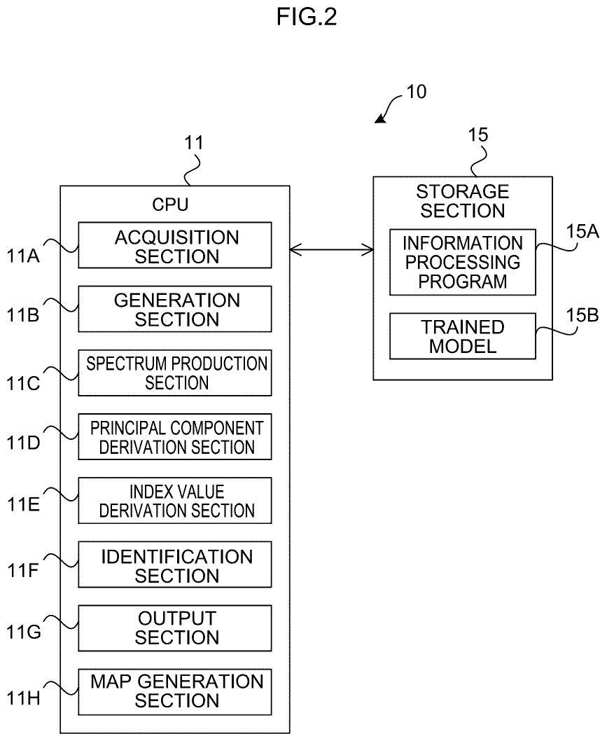

More specifically, the CPU 11 of the server 10 according to the present exemplary embodiment functions as each section illustrated in FIG. 2 by reading the information processing program 15A stored on the ROM 12 or the storage section 15, and executing the information processing program 15A in the RAM 13.

FIG. 2 is a block diagram illustrating an example of a functional configuration of the server 10 according to the present exemplary embodiment.

As illustrated in FIG. 2, the CPU 11 of the server 10 according to the present exemplary embodiment functions as an acquisition section 11A, a generation section 11B, a spectrum production section 11C, a principal component derivation section 11D, an index value derivation section 11E, an identification section 11F, an output section 11G, and a map generation section 11H.

The acquisition section 11A acquires molecule data files from the user terminal 30. Molecule data files are files stored with information representing molecule structures and, for example, molfiles may be employed therefor.

The generation section 11B generates atom-coordinate images expressing atomic coordinates in molecules. More specifically, the generation section 11B generates two-dimensional or three-dimensional atom-coordinate images from the molecule data files acquired by the acquisition section 11A. Information to indicate positions in a structure of atoms configuring a molecule is included in the molecule data files. The atom-coordinate images are, for example, generated based on positions in structures of atoms obtained from the molecule data files.

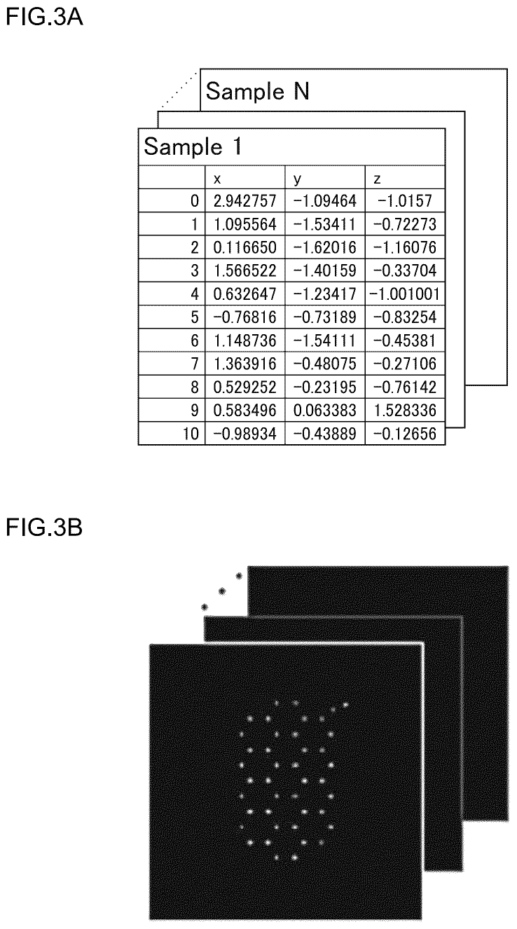

FIG. 3A is a diagram illustrating an example of molecule data files according to the present exemplary embodiment. FIG. 3B is a diagram illustrating an example of atom-coordinate images according to the present exemplary embodiment.

The molecule data files illustrated in FIG. 3A are, as an example, molfiles that correspond to molecules having a sample number of N. The atom-coordinate images illustrated in FIG. 3B have a single frame image corresponding to each single molecule of the molecule data files, with atoms represented by white points in the image.

The spectrum production section 11C performs a Fourier transformation on the atom-coordinate images generated by the generation section 11B so as to produce power spectrum data.

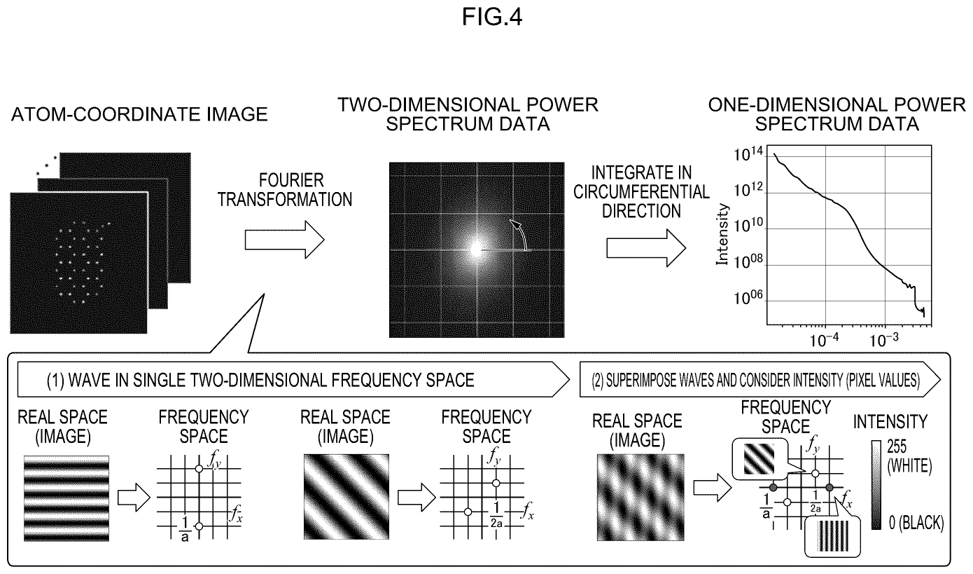

FIG. 4 is a diagram to accompany explanation of how a power spectrum is made of the atom-coordinate images according to the present exemplary embodiment.

As illustrated in FIG. 4, two-dimensional power spectrum data is produced by performing a Fourier transformation on the atom-coordinate images. In a Fourier transformation, waves are expressed in a single two-dimensional frequency space. Note that for three-dimensional atom-coordinate images, a three-dimensional Fourier transformation is performed to produce three-dimensional power spectrum data. Next, the two-dimensional power spectrum data is integrated in the circumferential direction to produce one-dimensional power spectrum data. Waves are superimposed by integrating in the circumferential direction and intensity (pixel values) are considered. Note that for three-dimensional power spectrum data, similarly to for two-dimensions, integration is also performed in the circumferential direction and one-dimensional power spectrum data is produced. In making a power spectrum, features of the images are expressed as “waves” by performing a Fourier transformation on the atom-coordinate image. This thereby enables periodic structure information to be obtained via “particle size”, “particle shape”, and “particle position” in the images.

The principal component derivation section 11D performs principal component analysis (PCA), which is a type of dimension reduction method, on the power spectrum data produced by the spectrum production section 11C, and derives the principal component vectors and the principal component scores from the power spectrum data. The principal component vectors represent basis vectors of the power spectrum data. The principal component vectors include respective components of spectrum values of the principal components. The principal component scores are feature values of the power spectrum data, and are coefficients expressing the contained quantities of the principal component vectors, namely how much is contained of the components of the principal component vectors.

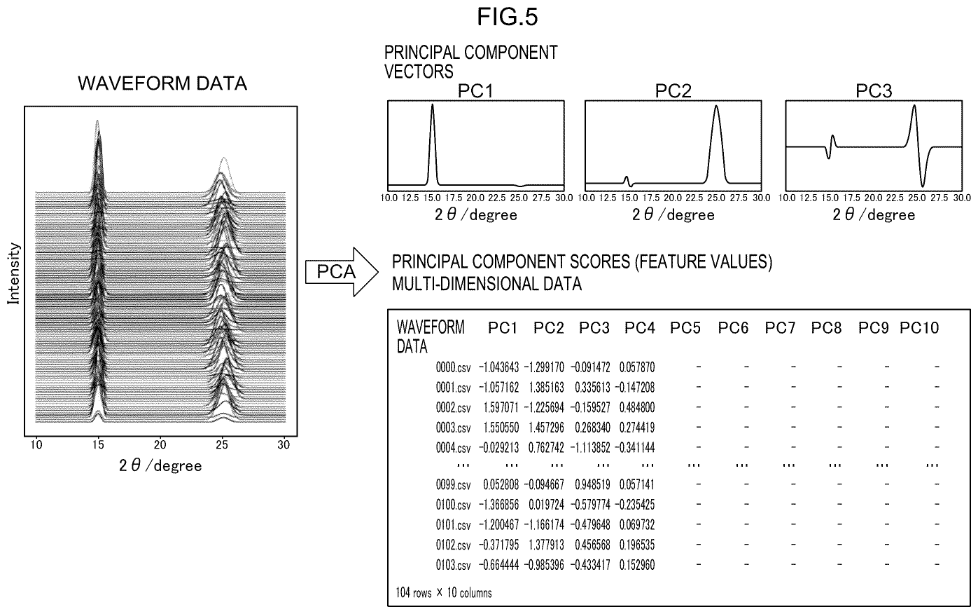

FIG. 5 is a diagram to accompany explanation of featurization of waveform data according to the present exemplary embodiment.

As illustrated in FIG. 5, principal component analysis (PCA) is performed on the waveform data, and the principal component vectors and the principal component scores are derived. The waveform data, for example for X-ray diffraction, is expressed by a diffraction angle (2θ (degrees)) on the horizontal axis and by intensity on the vertical axis. In the example of FIG. 5 there are ten individual principal component vectors derived. These ten individual principal components are called PC1 to PC10 below. The principal component vectors illustrated in FIG. 5 are the principal component vector of the first principal component PC1, the principal component vector of the second principal component PC2, and the principal component vector of the third principal component PC3.

However, the principal component scores illustrated in FIG. 5 are derived for each of the principal components PC1 to PC10 for each of the waveform data. Namely, the principal component scores indicate amounts contained of the principal component vectors for each of the principal components PC1 to PC10 for each of the plural waveform data.

The index value derivation section 11E derives index values expressing a degree of correlation between the principal component scores derived by the principal component derivation section 11D and molecule performance. The index value derivation section 11E may, for example, employ a learned model 15B stored on the storage section 15 so as to derive index values. The learned model 15B is a model that has undergone machine learning in advance so as to output molecule performance in response to being input with principal component scores. More specifically a configuration may be adopted in which plural sets of training data are prepared in which principal component scores have been associated with molecule performance, a learned model is generated based on the plural sets of training data, and the learned model is utilized to index values. In such a configuration, a known machine learning model may be employed as the learned model 15B. The learned model 15B may, for example, be generated by training the machine learning model using a deep learning algorithm. Note that in the present exemplary embodiment reference to “index values” means values indicating positive correlations and negative correlations to molecule performance. In the “index values”, for positive correlations the values are higher the higher the degree of contribution to a molecule performance, and for negative correlations the values are lower the higher the degree of contribution to the molecule performance. Moreover, reference to the “molecule performance” indicates a property or a capability processed by the molecule.

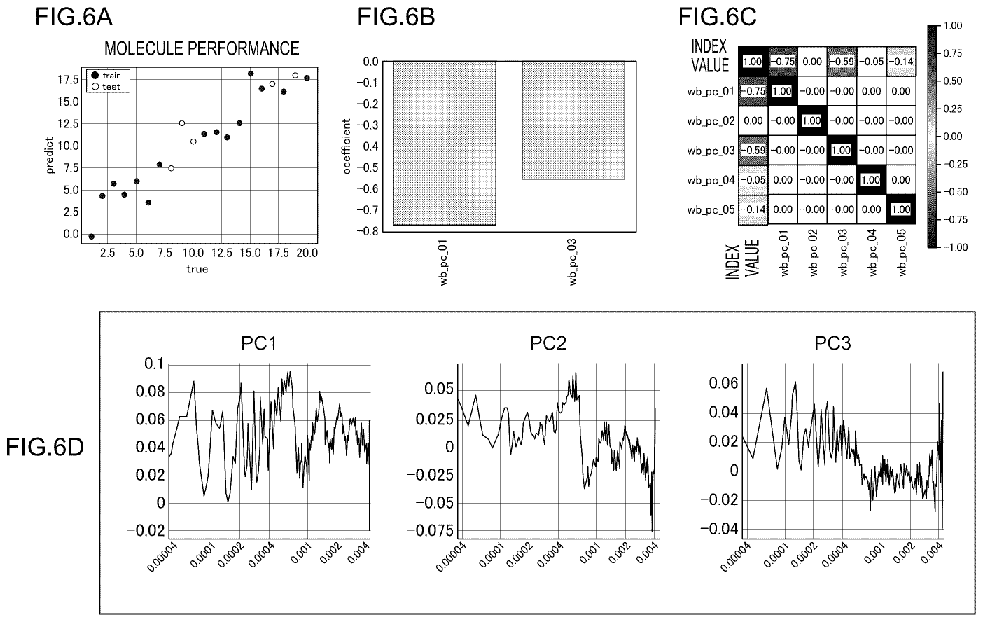

FIG. 6A is a graph illustrating an example of plot points plotting true values and prediction values related to molecule performance as output from the learned model 15B according to the present exemplary embodiment. The true values (true) are illustrated on the horizontal axis, and the prediction values (predict) are illustrated on the vertical axis.

As illustrated in FIG. 6A, machine learning of the learned model 15B employs, for example, training data (train) and test data (test). The training data (train) is data employed for training the machine learning model, and is data employed to update weightings of deep learning. The test data (test) is data to determine a level of performance of the learned model.

FIG. 6B and FIG. 6C are diagrams illustrating examples of index values derived for each of the principal component vectors by employing molecule performance obtained using the learned model 15B.

In the graph illustrated in FIG. 6B, “wb_pc_01”, “wb_pc_03” respectively represent “PC1” and “PC3”, and “coefficient” represents index values. Moreover, in the table illustrated in FIG. 6C, “wb_pc_01” to “wb_pc_05” respectively represent “PC1” to “PC5”. Note that “PC6” to “PC10” have been omitted in order to simplify explanation. As described above, the index values include both positive correlations and negative correlations and are expressed, for example, in a range of from “−1.00” to “1.00”. Namely, higher positive correlations approach “1.00”, and higher negative correlations approach “−1.00”. For example, the index value of the “PC1” is “4.75”, the index value of the “PC3” is “−0.59”, and it is apparent that these are both high negative correlations. The graph of FIG. 6B results from making a graph of the index values of the “PC1” and “PC3” of FIG. 6C.

The identification section 11F identifies principal component vectors based on the index values derived by the index value derivation section 11E. More specifically, the identification section 11F, for example, identifies any principal component vectors for which the absolute value of the index value derived by the index value derivation section 11E is a threshold or greater. Note that the threshold may be set as an appropriate value based on experimentation or based on historical knowledge. More specifically, for example, “0.5” is set as the threshold for the index values illustrated in FIG. 6C. In this cases the absolute values of the index values of “PC1” and “PC3” (respectively “0.75” and “0.59”) are both the threshold or greater, and so from out of the principal components PC1 to PC10 the principal components PC1 and PC3 are identified as being principal component vectors having a high degree of contribution to the molecule performance.

The output section 11G outputs principal component power spectrum data that is power spectrum data corresponding to the principal component vectors identified by the identification section 11F. Note that in order to discriminate from the above power spectrum data of molecules, the power spectrum data corresponding to the principal component vectors is called the principal component power spectrum data. The principal component power spectrum data is obtained by performing the principal component analysis described above. The output section 11G outputs the principal component power spectrum data to, for example, the display section 32 of the user terminal 30.

FIG. 6D includes graphs illustrating an example of principal component power spectrum data corresponding to the principal component vectors. The principal component power spectrum data corresponding to the principal component vectors of “PC1” to “PC3” are illustrated here, as an example. The horizontal axis of the graph of the principal component power spectrum data indicates, for example, frequency and the vertical axis thereof indicates the spectrum values.

The output section 11G displays, on the display section 32 of the user terminal 30, the principal component power spectrum data of the “PC1 and “PC3” that have been identified as principal components having a high degree of contribution to molecule performance from out of the principal component power spectrum data for “PC1” to “PC3” illustrated in FIG. 6D. This thereby enables a user to understand a shape of the principal components having a high degree of contribution to molecule performance.

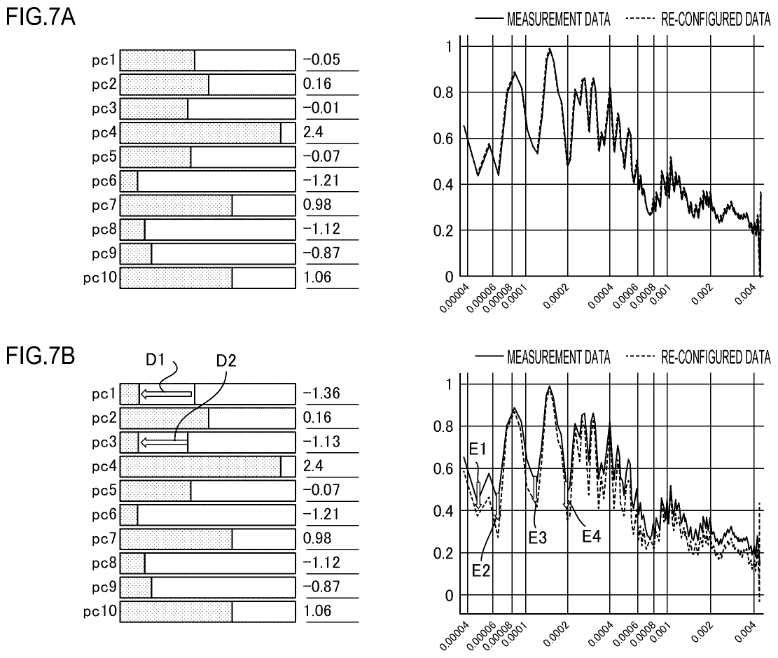

FIG. 7A is a graph illustrating an example of power spectrum data prior to changing the principal component scores of the principal components PC1 and PC3. FIG. 7B is a graph illustrating an example of power spectrum data after changing the principal component scores of the principal components PC1 and PC3. The solid lines in FIG. 7A and FIG. 7B are of measurement data of the power spectrum, and the doted lines therein are of re-configured data obtained using the principal component vectors and the principal component scores.

As illustrated in FIG. 7A and FIG. 7B, when the principal component scores are reduced for the principal components PC1 and PC3 that are principal component having a high degree of contribution to the molecule performance, the power spectrum data of the whole molecule including the principal components PC1 to PC10 is also changed. When this is performed, a molecule having atomic coordinates corresponding to the changed shape of the spectrum can be understood as being a molecule having high performance.

Note that the principal component scores displayed by bars in FIG. 7A and FIG. 7B are preferably adjustable by the user terminal 30. For example, a configuration may be adopted in which the principal component scores are changed as indicated by arrows D1, D2 illustrated in FIG. 7B such that a waveform of the power spectrum data is changed so as to become that of the arrows E1 to E4. In such cases the waveform of the power spectrum data is changed according to change adjustments of the principal component scores being displayed by bars, enabling the user to understand the meaning of the principal component scores.

The map generation section 11H generates two-dimensional map data of the principal component scores corresponding to each of the plural principal component vectors identified by the identification section 11F projected as plural plot points onto two-dimensions. In such cases a configuration may be adopted in which in which the output section 11G displays the two-dimensional map data together with an atom-coordinate image corresponding to plot points of the two-dimensional map data on the display section 32 of the user terminal 30.

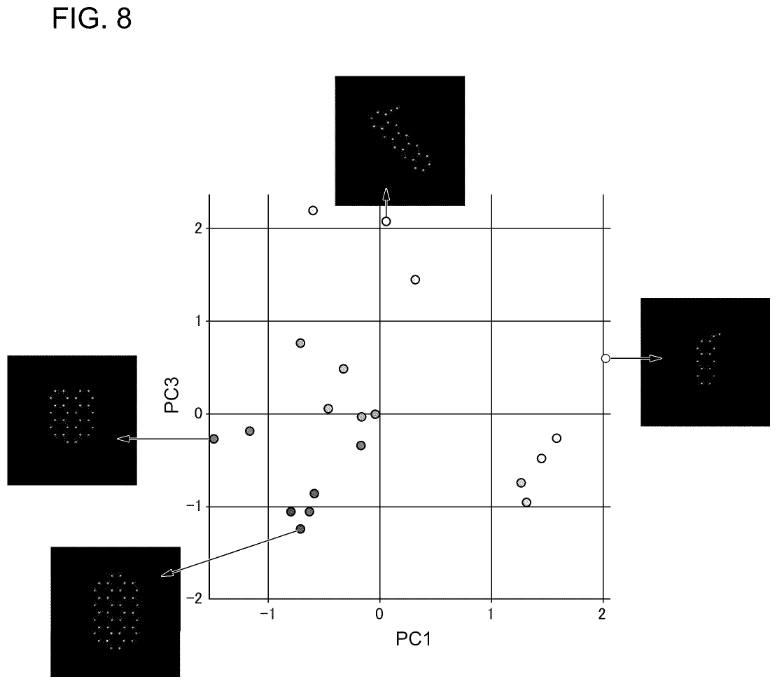

FIG. 8 is a diagram illustrating an example of two-dimensional map data and atom-coordinate images according to the present exemplary embodiment.

The two-dimensional map data illustrated in FIG. 8 is data resulting from projecting the principal component scores corresponding to each of the principal components PC1 and PC3 identified by the identification section 11F as plural plot points onto two-dimensions. The horizontal axis indicates the principal component PC1, and the vertical axis indicates the principal component PC3.

In FIG. 8, coordinate values of a single plot point of the two-dimensional map data corresponds to the principal component scores of the principal components PC1 and PC3. As stated above, the waveform of the power spectrum data is changed according to the principal component scores of the principal components PC1 and PC3. In other words, due to the power spectrum data being decided corresponding to the plot points, the power spectrum data can be transformed to obtain atom-coordinate images. The transformation from the power spectrum data into the atom-coordinate images may, for example, be achieved by performing an inverse Fourier transformation, or may be performed using machine learning. Thus, for example, when a user hypothesizes a molecule having a desired performance, an atom-coordinate image of this unknown molecule can be obtained without actually imaging the unknown molecule expected to have this performance.

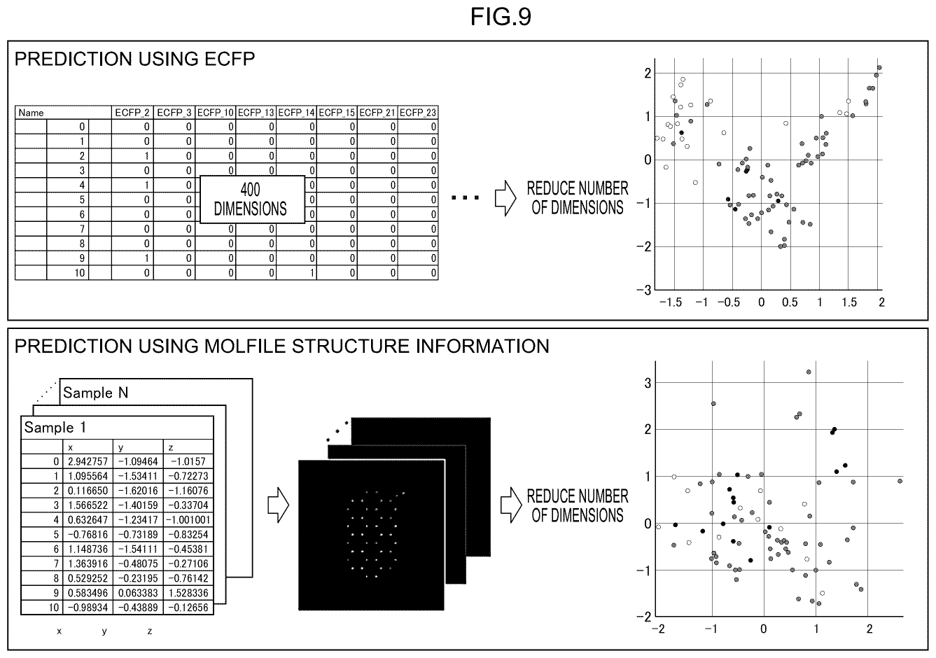

FIG. 9 is a diagram to accompany explanation of prediction using the molfile structure information according to the present exemplary embodiment.

As illustrated in FIG. 9, when a prediction is made using the molfile structure information according to the present exemplary embodiment, the molecule can be represented by fewer feature values than when prediction uses ECFP according to a comparative example. The computation load is accordingly reduced when searching for molecules.

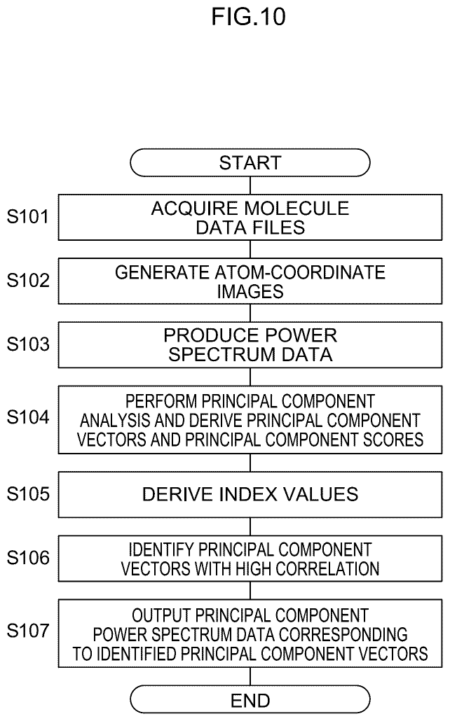

Next, description follows regarding an operation of the server 10 according to the present exemplary embodiment, with reference to FIG. 10.

FIG. 10 is a flowchart illustrating an example of a flow of processing by the information processing program 15A according to the present exemplary embodiment.

First, when execution of molecule search processing is instructed to the server 10, the information processing program 15A is started up by the CPU 11, and each of the following processing is executed.

At step S101 of FIG. 10, the CPU 11 acquires, as an example, the molecule data file as described above illustrated in FIG. 3A from the user terminal 30.

At step S102 the CPU 11 generates, as an example, the atom-coordinate images illustrated in above FIG. 3B from the molecule data file acquired at step S101.

At step S103 the CPU 11 performs, as an example, a Fourier transformation on the atom-coordinate images generated at step S102 to produce power spectrum data as illustrated in FIG. 4.

At step S104 the CPU 11 performs, as an example, principal component analysis on the power spectrum data produced at step S103, as illustrated in FIG. 5, and derives the principal component vectors and the principal component scores from the power spectrum data.

At step S105 the CPU 11 employs, as an example, the learned model 15B to derive the index values expressing degrees of correlation between the principal component scores derived at step S104 and molecule performance.

At step S106 the CPU 11 identifies, as an example, principal component vectors for which the absolute values of the index values derived at step S105 are a threshold or greater, as illustrated in FIG. 6C.

At step S107 the CPU 11 outputs, as an example, the principal component power spectrum data corresponding to the principal component vectors identified at step S106, as illustrated in FIG. 6D, to the display section 32 of the user terminal 30, and then one cycle of processing by the information processing program 15A is ended.

As described above, the present exemplary embodiment enables a possibility of discovering an unknown molecule that satisfies a performance condition to be raised compared to cases in which a comparison is made against a database.

Moreover, molecules can be represented with fewer feature values than in prediction using ECFP. This accordingly enables a reduction in computation load when searching for molecules.

Note that “processor” in the above exemplary embodiment indicates a wide definition of processors, and encompasses general purpose processors (such as central processing units (CPU) and the like), and custom processors (such as graphics processing units (GPU), application specific integrated circuits (ASIC), field programmable gate arrays (FPGA), programmable logic devices, and the like).

Moreover, each of the actions of the processor in the exemplary embodiment is not necessarily achieved by a single processor alone, and may be achieved by cooperation between plural processors present at physically separated locations. Moreover, the sequence of each of the actions of the processor is not limited to the sequence described in the above exemplary embodiment, and may be rearranged as appropriate.

Explanation has been given regarding an example of an information processing device according to an exemplary embodiment. The exemplary embodiment may be provided in the format of a program configured to cause a computer to execute the functions of the information processing device. The exemplary embodiment may be provided in the format of a computer-readable non-transitory storage medium stored with such a program.

Configurations of the information processing device described in the above exemplary embodiment are moreover merely examples thereof, and may be modified according to circumstances within a range not departing from the spirit thereof.

The processing flow of the program described in the above exemplary embodiment is moreover also merely an example thereof, and redundant steps may be omitted, new steps may be added, or the processing sequence may be altered within a range not departing from the spirit of the present disclosure.

Although explanation in each of the above exemplary embodiment is regarding a case in which the processing according to the exemplary embodiment is implemented by a software configuration employing a computer by execution of a program, there is no limitation thereto. For example, an exemplary embodiment may be implemented by a hardware configuration, or by a combination of a hardware configuration and a software configuration.

What is claimed is:

1. An information processing device, comprising:
a memory, and
a processor coupled to the memory,
wherein the processor is configured to:
generate an atom-coordinate image expressing atomic coordinates in a molecule;
perform a Fourier transformation on the atom-coordinate image to produce power spectrum data;
perform principal component analysis on the power spectrum data so as to derive, from the power spectrum data, principal component vectors expressing basis vectors of the power spectrum data and principal component scores expressing contained quantities of the principal component vectors;
derive index values expressing degrees of correlation between the principal component scores and a performance of the molecule;
identify any principal component vectors that correlate with the molecule performance based on the index values; and
output principal component power spectrum data that is power spectrum data corresponding to the principal component vectors.

2. The information processing device of claim 1, wherein the processor is configured to derive the index values using a learned model that has undergone machine learning in advance so as to output the molecule performance in response to being input with the principal component scores.

3. The information processing device of claim 1, wherein the processor is configured to generate the atom-coordinate image in two-dimensions or in three-dimensions, from a molecule data file stored with information representing a structure of the molecule.

4. The information processing device of claim 1, wherein the processor is configured to identify a plurality of principal component vectors, and to generate two-dimensional map data in which the principal component scores corresponding to each of the plurality of principal component vectors are projected as a plurality of plot points onto two-dimensions.

5. The information processing device of claim 4, wherein the processor is configured to display the two-dimensional map data together with the atom-coordinate image corresponding to the plot points of the two-dimensional map data at a display.

6. An information processing method, comprising
an information processing device:
generating an atom-coordinate image expressing atomic coordinates in a molecule;
performing a Fourier transformation on the atom-coordinate image to produce power spectrum data;
performing principal component analysis on the power spectrum data so as to derive, from the power spectrum, data principal component vectors expressing basis vectors of the power spectrum data and principal component scores expressing contained quantities of the principal component vectors;
deriving index values expressing degrees of correlation between the principal component scores and a performance of the molecule;
identifying any principal component vectors that correlate with the molecule performance based on the index values; and
outputting principal component power spectrum data that is power spectrum data corresponding to the principal component vectors.

7. A non-transitory computer readable storage medium storing an information processing program that causes a computer to execute processing, the processing comprising:
generating an atom-coordinate image expressing atomic coordinates in a molecule;
performing a Fourier transformation on the atom-coordinate image to produce power spectrum data;
performing principal component analysis on the power spectrum data so as to derive, from the power spectrum data, principal component vectors expressing basis vectors of the power spectrum data and principal component scores expressing contained quantities of the principal component vectors;
deriving index values expressing degrees of correlation between the principal component scores and a performance of the molecule;
identifying any principal component vectors that correlate with the molecule performance based on the index values; and
outputting principal component power spectrum data that is power spectrum data corresponding to the principal component vectors.
